(19) United States
(12) Patent Application Publication
(10) Pub. No.: US 2007/0261980 A1
(43) Pub. Date: Nov. 15, 2007

(54) Package

(76) Inventors: Mark Anthony Cox, Hertfordshire (GB); Timothy James Uden, London (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/570,395

(22) PCT Filed: Jun. 30, 2005
(86) PCT No.: PCT/GB05/02549

§ 371 Date: Dec. 11, 2006

(30) Foreign Application Priority Data
Jul. 2, 2004 (GB) 0414984.5

(51) Int. Cl. B65D 5/50; A61B 19/02; B65B 7/00

(52) U.S. Cl. 206/438; 53/476

(57) ABSTRACT

A package in which a dispenser is contained and having at least one support which suspends the dispenser in the package is disclosed. Suspending the dispenser within the package protects it from impacts and deformations of the package, which may otherwise cause it to activate during transit, or if it is provided with a dose-counter, miscount the number of remaining or dispensed.

### FIELD OF INVENTION

The present invention relates to a package for a dispensing device.

The invention is particularly, but not exclusively, concerned with a package for a drug-dispensing device such as an inhaler and especially a pressurised metered dose inhaler (hereinafter referred to as a “pMDI”). The invention does, however, embrace other dispenser types, for example a dry powder inhaler (DPI), as will be appreciated by the reader skilled in the inhaler art.

### BACKGROUND OF THE INVENTION

pMDIs are well known in the art of inhalation devices. It is therefore not necessary to describe the construction and operation of a pMDI other than in bare essentials.

A pMDI comprises a canister unit and a housing. The housing is generally tubular, although this is not essential, and generally formed of a plastics material, for instance by moulding. The canister unit comprises a canister having one open end, typically made from a metal such as aluminium. The open end of the canister is sealingly capped by a metering valve assembly. The valve assembly includes a hollow dispensing member, usually in the form of a valve stem, which projects from the open end of the canister. The dispensing member is mounted for sliding movement relative to the canister between an extended position, to which the dispensing member is biased by a biasing mechanism in the valve assembly, and a depressed position. Movement of the dispensing member from the extended position to the depressed position results in a metered dose of the aerosol formulation being dispensed from the canister through the dispensing member. In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may, for example, be one of the hydrofluoroalkanes (HFA) 1,1,1,2-tetrafluoroethane (HFA-134a) and 1,1,1,2,3,3,3-heptafluoropropane (HFA-227).

The housing comprises an internal passageway having an open end. The canister unit is slidable into the internal passageway through the open end with the canister unit being inserted valve assembly first into the internal passageway. A stem block, which receives the dispensing member of the canister when the canister unit is received in the housing in a rest position, has a passageway with an inlet end for receiving the dispensing member and an outlet end, which faces a dispensing outlet of the housing, typically a mouthpiece or a nasal nozzle. The stem block holds the dispensing member stationary whereby depression of the canister unit from its rest position further into the housing to an actuated position causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister unit from the rest position to the actuated position. The inspiratory airflow produced by the patient entrains the metered dose of the medicinal aerosol formulation into the patient's respiratory tract.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or another respiratory condition, as a result of which the inhalation of foreign material may cause severe irritation.

Developments to pMDIs have included the provision of actuation indicators or dose counters therefor. Such a dose counter is described in PCT Patent Applications Nos. WO-A-9856444 and WO-A-2004/001664 to Glaxo Group Limited. The pMDI canister unit may comprise the dose counter, which is fixably secured on the valve assembly end of the canister and includes a display which denotes the number of metered doses of the medicament formulation dispensed from, or remaining in, the canister. The display of the dose counter may be visible to the patient through a window provided in the housing. The display may be presented by a plurality of indicator wheels rotatably mounted on a common axle, each wheel having numerals from ‘0’to ‘9’ displayed in series around the circumference.

pMDI devices, however, are susceptible to unintentional actuation, particularly whilst in transit, for example shipment between the manufacturer and distributor. During such transit, such devices and their package are often subjected to impacts and sudden movements. Such forces can actuate the pMDI, causing doses of the formulation to be dispensed. When the pMDI includes a dose counter, rough handling in transit can cause the value displayed to the user by the counter to increase or decrease so that it is not consistent with the number of doses that have been dispensed by, or remain in, the pMDI. It is wasteful to dispense unused doses of the medicament, and potentially very dangerous for a dose counter to indicate to the user that more doses remain in the canister than are actually present.

pMDIs are ordinarily stored in a sealed overwrap inside a cardboard box. The overwrap is commonly made from foil, for example, and protects the pMDI from moisture. WO-A-2001/87392 A2 describes a sealed overwrap that prevents moisture ingress but is permeable to the propellant gas of the pMDI contained therein. This prevents the overwrap from bursting if its interior pressure increases due to leakage of the propellant from the container. A desiccant is also provided inside the overwrap.

WO-A-2004/002559 describes a sealed overwrap for an HFA-propelled pMDI comprising a quantity of HFA-absorbent material to absorb any leaked propellant to maintain the interior of the package at ambient pressure.

However, these prior art packages do not prevent unintentional actuation of the pMDI. If a pMDI-containing package is roughly handled the pMDI is free to move inside the sealed packed and impact surfaces exterior to the package, for example the walls of an exterior package such as a cardboard box.

Unintentional actuation of the inhaler in a prior art package can happen in one of a number of ways. For example, a sudden acceleration of the outer package may cause the inhaler to move relative to it and strike an interior wall. In the exemplary case of a pMDI this impact of the pMDI against the wall may be sufficient to cause the canister unit to be depressed within the housing, causing a dose to be dispensed and/or a dose counter to increment or decrement. Alternatively, the initial acceleration itself may be sufficient to cause the container unit to move within the housing with sufficient momentum for actuation to occur. In a third possibility, the outer package may become deformed as a result of mishandling and the deformed area of the package may press against the pMDI, causing it to be dispensed. Although the above illustrations are made with reference to pMDI having a housing and a canister unit, it will be understood that other inhaler types may be similarly unintentionally actuated.

This is highly problematic since, for example, during actuation, the pMDI dispenses medicament formulation at a higher rate than can be absorbed by a propellant-absorbent insert or escape through the ‘breathable’ material of the prior art overwrap. Consequently, if a current pMDI-containing package is roughly handled propellant will build-up inside the package and, if the pressure is sufficient and the package is sealed, burst it.

Other than the possibility of bursting, there are clearly other disadvantages to current packages. Accidental actuation during transit is wasteful and leaves the consumer with fewer remaining doses at the time of purchase. Also, impacts can cause some prior art dose counters to miscount, or the pMDI to ‘partially actuate’, where the shock is sufficient to cause the pMDI to dispense at least a partial dose of the medicament formulation, but insufficient to update the dose counter. This latter problem is dangerous for the user, as discussed above.

Therefore, it is clearly desirable to provide a package for a dispensing device that prevents its unintentional actuation. It is also desirable to provide a package for a dispenser that prevents the dose counter of the dispenser from miscounting the number of dispensed doses.

### STATEMENTS OF INVENTION

A first aspect of the present invention provides a package containing a dispenser and at least one support which suspends the dispenser in the package. Preferably, the package is provided with an outer package in which the dispenser is suspended by the support. The dispenser may be operated to dispense by the relative motion of its parts and may be an inhaler—particularly, but not exclusively, a pMDI.

This is particularly advantageous over prior art packages, since the suspension of the dispenser reduces or eliminates contact between the dispenser and the package.

The present invention substantially prevents the above-mentioned forms of unintentional actuation. Suspension of the dispenser within the package prevents it from being thrown about within the package during impacts and it cannot, therefore, strike the internal walls. Also, minor deformations of the package are unlikely to contact the dispenser, since it can be suspended away from the walls.

Preferably, the at least one support is made from a resilient material such as cardboard. Thus, the supports may structurally reinforce, and help to prevent deformation of, the package. Also, the supports dampen the effect of any sudden impact, since they flex in the event of an impact, absorbing some of the shock.

The at least one support may comprise at least one insert and may be in a deformed, including folded, configuration, in which case it may biased to return to a non-deformed configuration.

In a preferred embodiment, the support is deformed and has a generally semi-cylindrical form. Securing means are provided for securing the dispenser to the support. These securing means may comprise a tab for securing the top of the dispenser and a shelf for securing the base of said dispenser. The support of this embodiment may also have a generally ‘V’ shaped cross section. Finally, an end flap connecting the sides of the support at one or both ends may be present. The end flap not only prevents the insert from returning to its flat configuration, allowing it to be used with packages that are not sufficiently rigid, or appropriately dimensioned, to hold the support in its folded configuration, but may also be arched away from the insert in order to cushion the dispenser from impacts to the ends of the package.

In a first alternative embodiment a support is provided that is deformed into a generally ‘Z’ shape. The support comprises a window, through which, when packaged, the dispenser extends. The dispenser may be an interference fit in the window, to secure it in place in the insert. Shelves may be provided in the insert above and/or below the dispenser to further restrict its movement.

In a second alternative embodiment, the support comprises at least two flexible webs positioned on opposite sides of the dispenser. The opposite ends of each flexible web are positioned at opposite points on the interior surface of the package. The length of each web is preferably greater than the distance between the two opposite points between which it is positioned, so that it arches between the points. The dispenser is placed between the arched webs and is gripped between them. The webs may arch convexly or, alternatively, concavely. The webs may be inserts or, if the package is a container such as a box or carton or the like, may be formed by elongation of a flap of the container.

A second aspect of the invention provides a method of packaging a dispenser comprising providing an package and at least one support as defined in any one of claims 1 to 35; suspending the dispenser in the package with the support; and closing the package.

Further aspects and features of the present invention are set forth in the accompanying claims and in the exemplary embodiments described hereinafter with reference to the accompanying figures and drawings.

### BRIEF DESCRIPTION OF THE DRAWINGS

FIG. 1 shows a deformable insert for a package for a pMDI, according to an embodiment of the present invention;

FIG. 2 shows a pMDI secured by the insert of FIG. 1;

FIG. 3A and FIG. 3B show the insert and pMDI of FIG. 2 in an outer package;

FIG. 4 shows a deformable insert for a package for a pMDI, according to a another embodiment of the present invention, wherein the insert is provided with securable end flaps that can be used to hold it in its folded configuration;

FIG. 5 shows a pMDI secured by the insert of FIG. 4;

FIG. 6 shows the insert and pMDI of FIG. 5 in an overwrap;

FIG. 7 shoes an insert for a package for a pMDI, according to another embodiment of the present invention, securing a pMDI and an overwrap, wherein the pMDI is sealed inside the overwrap and supported between two shelves

FIG. 8 shows an insert for a package for a pMDI, according to another embodiment of the present invention, wherein the insert has a generally ‘Z’ shape;

FIG. 9 shows a pMDI secured by the insert of FIG. 8, wherein the pMDI is optionally sealed inside an overwrap;

FIG. 10A shows the insert of FIG. 8 securing a pMDI and FIG. 101B shows the insert and pMDI inside an outer package.

FIG. 11 shows two webs suspending a pMDI, optionally sealed within an overwrap, within an outer package, according to another embodiment of the present invention;

FIG. 12 shows an outer package comprising a box with extended flaps; and

FIG. 13 shows a pMDI, optionally in an overwrap, suspended in the outer package of FIG. 10, according to another embodiment of the present invention.

### DETAILED DESCRIPTION

In the following description, like reference numerals have been used to indicate like parts in the different embodiments of the invention. Each embodiment is adapted for use with a pMDI which is hand-held and hand-operable.

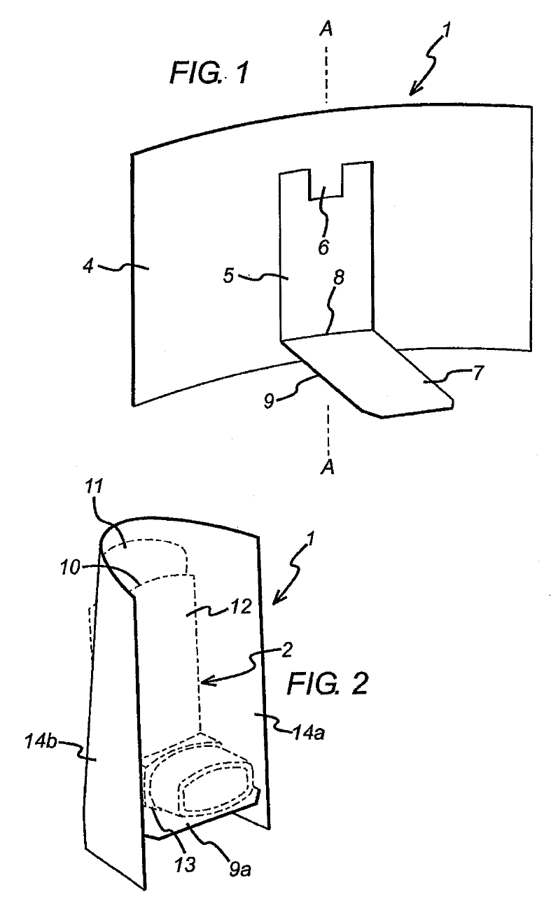

FIGS. 1 to 3B illustrate a first embodiment of the present invention. The embodiment comprises an insert (1) for supporting the pMDI (2) inside the outer package. The insert (1) is preferably manufactured from cardboard, since this is inexpensive, light, recyclable, resilient and pliable. The pliable nature of cardboard permits it to flex, absorbing light and moderate impacts, and to crumple when absorbing more severe impacts.

The insert (1) comprises a sheet (4) of resilient material, preferably cardboard, into which a window (5) is cut, leaving a tab (6) and a flap (7) at opposite ends of the window (5). The flap (7) is foldable about a score-line (8) and is folded generally perpendicular to the rest of the sheet (4) to form a first shelf (9a). As shown in FIG. 1, the sheet (4) is bent into a U-shape about an axis (A), although it could be folded or bent along the axis (A) into other shapes, for example a V-shape.

FIG. 2 shows the insert (1) of FIG. 1, to which a pMDI (2) has been removably attached by positioning the pMDI (2) inside the window (5) in the sheet, inserting the tab (6) between the interior surface of the open end (10) of the housing (12) and the container (11) and resting the opposite end of the housing (12), its ‘base’ (13), on the first shelf (9a). The tab (6) prevents the pMDI (2) from moving upward or from side to side and the first shelf (9a) prevents the pMDI (2) from moving downward.

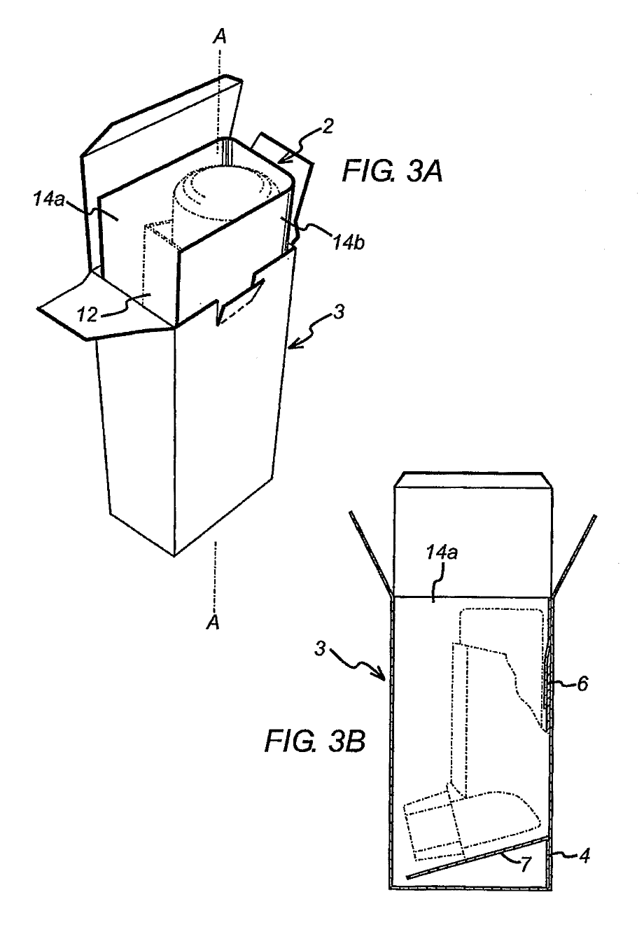

FIG. 3A and FIG. 3B shows the insert (1) and pMDI (2) of FIG. 2 in use, inserted into a cardboard box (3), although other outer packages, for example a heat-sealable overwrap, may be used. The material of the sheet (4) is resilient and is biased to return to its flat configuration and therefore the sides (14a, 14b) of the insert are forced against the interior walls of the box (3), holding it in position inside. Additionally, the length of the insert (1) is preferably matched to the length of the box (3), in order to restrict movement of the insert (1) along its axis (A) within the box. Thus, movement of the pMDI (2) relative to the box (3) is substantially prevented and a package according to the present invention is provided.

Of course, the assembly of the insert (1) and pMDI (2) could be sealed in an overwrap and then inserted into the cardboard box (3).

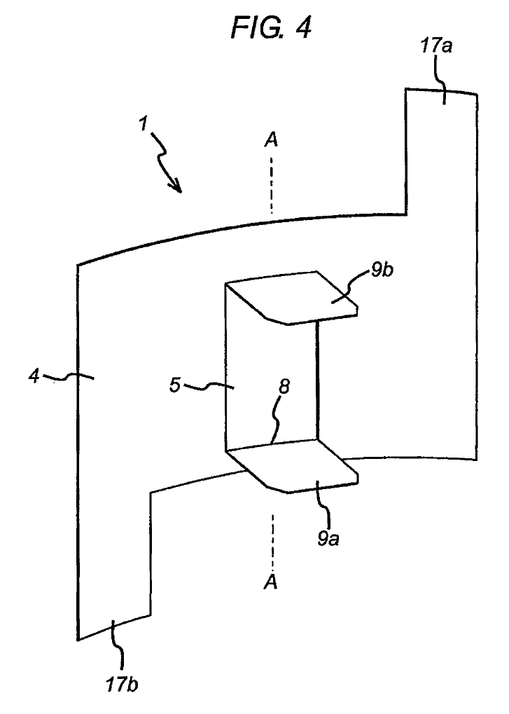

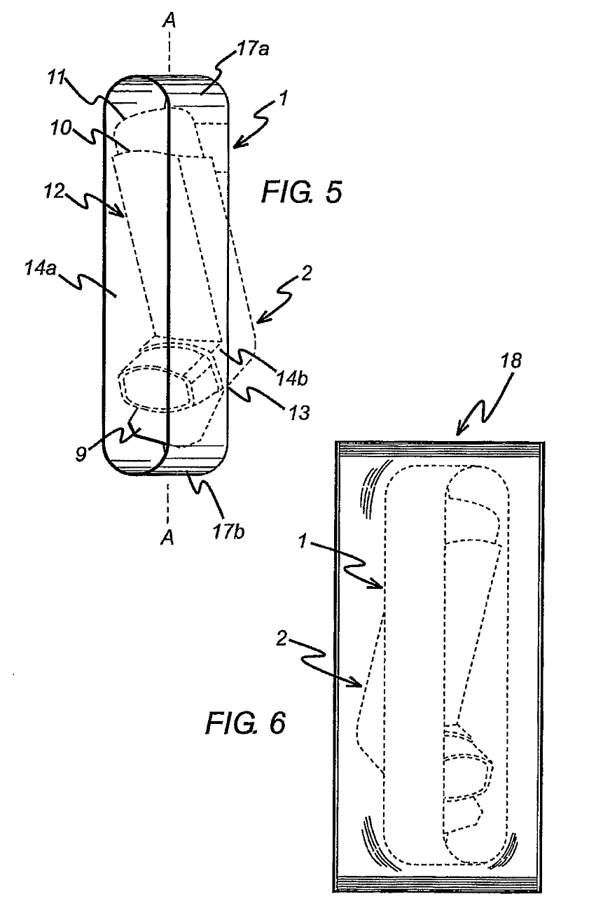

FIG. 4 illustrates a second embodiment of the present invention, which is similar to the previously described embodiment except that the tab (6) is replaced by a second shelf (9b) and the insert (1) further comprises flaps (17a, 17b), which can be folded or bent over the top and bottom of the insert (1) where they are secured in place, for example by glue. In FIG. 4, the insert (1) is shown folded into a generally U-shape about axis A, but it may be bent or folded into other shapes. The flaps (17a, 17b) prevent the sheet (4) from returning to its unfolded configuration. This allows the insert (1) to be inserted into an outer package that would otherwise lack the rigidity to keep the insert folded. The flaps (17a, 17b) are preferably arched, as shown in FIG. 5, or V-shaped, and elastically deformable in order to dampen impacts.

In an alternative embodiment, the tab (6) is not replaced by a second shelf, but the flaps (17a and 17b) are present. FIG. 5 illustrates a pMDI (2) secured by such an arrangement and FIG. 6 shows the pMDI (2) and insert (1) of FIG. 5 inserted into an overwrap (18).

The overwrap (18) may be made from a heat-sealable foil or plastic which is then heat-sealed with the insert (1) and pMDI (2) inside it, providing a package according to the present invention. The use of such an overwrap (18) as an outer package reduces the total weight and cost of the overall package, and may be made impermeable to water vapour and air, so that the pMDI (2) is hermetically sealed in the package. The overwrap (18) and its contents can be placed in a further container, for example a box, optionally using another suspending support of the invention.

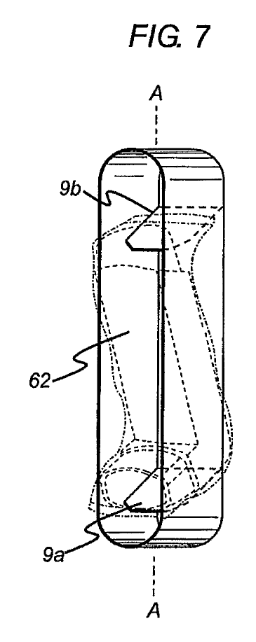

FIG. 7 shows a pMDI (2) inside an overwrap (62), secured by the insert (1) of FIG. 4. The pMDI (2) and overwrap (62) are supported by the first and second shelves (9a and 9b).

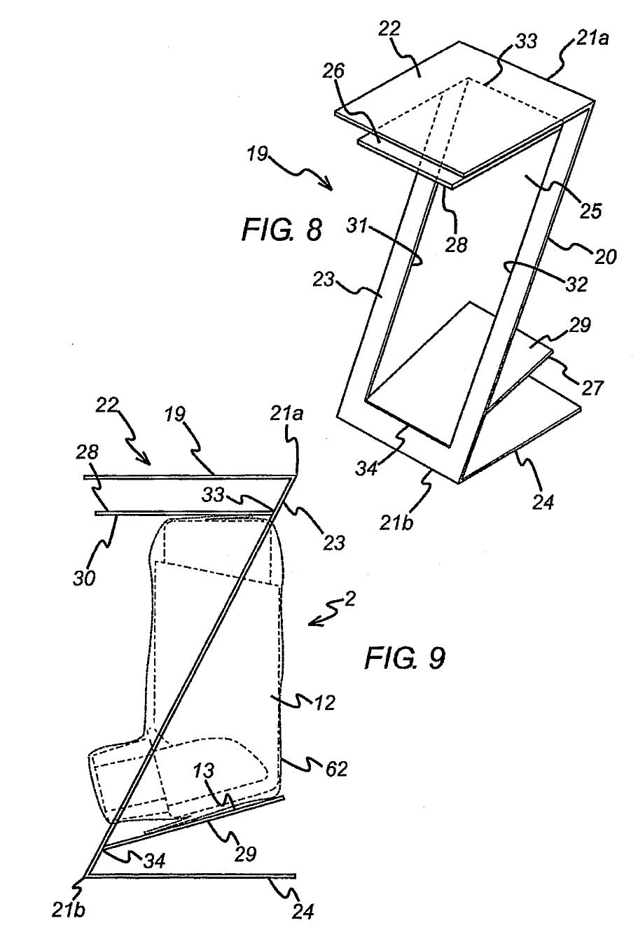

FIG. 8 shows an insert (19) for use in another embodiment of the present invention. A sheet (20) of cardboard is folded, in opposite directions about score lines (21a, 21b) located across the sheet at separated points along its length, into a Z-shape. The folded insert (19) has three portions, a top portion (22), a middle portion (23) and a bottom portion (24), wherein the top (22) and bottom portions (24) are at opposite ends of the middle portion (23). A window (25) is provided in the middle portion (23), the window (25) being marginally narrower than the pMDI (2) so that, when inserted into the window (25), the pMDI (2) is an interference fit.

Optionally, the window (25) is formed by cutting the middle portion (23) of the sheet to provide first and second flaps (26, 27), foldable about opposite edges (33, 34) of the window. The first flap (26) is folded away from the plane of the middle portion (23) to form a shelf (28) on one side of the middle portion (23) and the second flap (27) is folded in the opposite direction to form a second shelf (29) on the opposite side of the middle portion (23).

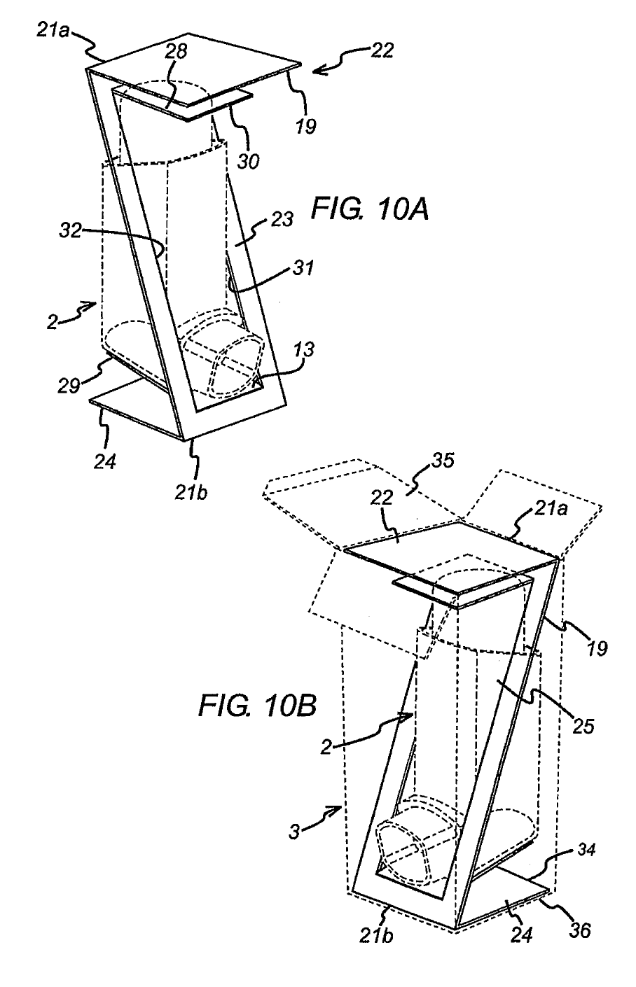

FIGS. 9 and 10A illustrate the insert (19) of FIG. 8, with first and second shelves (28, 29), in use. A pMDI (2) is inserted into the window (25) so that the top portion (22) overlies the top (30) of the pMDI (2) and the bottom portion (24) underlies the base (13) of the pMDI (2). When the pMDI (2) is positioned in the window (25), the first shelf (28) overlies and supports its top (30) and the second shelf (29) underlies and supports its base (13). Thus, the pMDI (2) is held securely between the side edges (31, 32) of the window and between the two shelves (28, 29). As shown in FIG. 9, the pMDI (2) may be inserted into an overwrap (62), and the pMDI (2) and overwrap (62) both inserted into the window (25) supported between the shelves (28, 29). The shelves (28, 29) are capable of limited motion about their fold lines (33, 34), which dampens the effect of impacts.

In use, the insert (19), with the pMDI (2) attached, is placed in an outer packaging. FIG. 10B shows the insert in a cardboard box (3), although other outer packages could be used. As the insert (19) attempts to return to its unfolded configuration, its top and bottom portions (22, 24) are forced against opposite faces of the box (35, 36) securing the insert (19) within the box (3).

In another embodiment of the present invention, the support comprises at least one arched web ((37) or (38)), made from a resilient material. Preferably, the resilient material is cardboard and the support comprises a first and second web (or a single web folded into two portions).

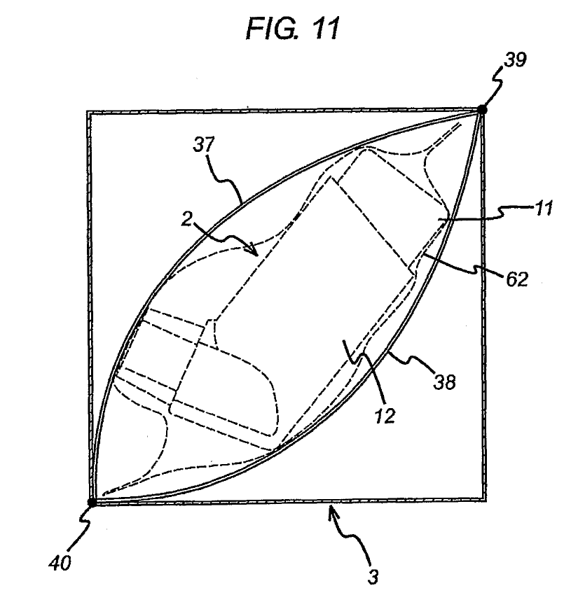

Each web has first and second ends that meet the inner surface of the outer package at first and second locations. The length of the web is greater than the distance between these first and second locations, and the web arches between them. FIG. 11 shows a preferred embodiment wherein two webs (37, 38) are provided with common first and second locations (39, 40).

The webs of FIG. 11 are positioned within a cuboid box (3), such that the first and second common locations (39, 40) correspond to diagonally opposite interior edges of the box (3). The pMDI (2) is inserted between the webs (37, 38), their resilience causing it to be gripped between them and suspended within the box (3). The pMDI (2) is thus substantially prevented from movement within the box (3) and cushioned, by the webs (37, 38), from impact. Although a cuboid box (3) is preferable, it is obvious to those skilled in the art that other outer packages could be used.

The pMDI (2) may be in an overwrap (62), wherein the overwrap (62) is held suspended by the webs (37, 38).

The webs may be formed as an insert having a purse configuration, as illustrated in FIG. 11. The ends of the purse are pushed towards each other, deforming the purse and allowing the pMDI (2) (optionally inside an overwrap (62)) to be inserted between the webs (37, 38). The insert and pMDI (2) are then inserted into the box (3).

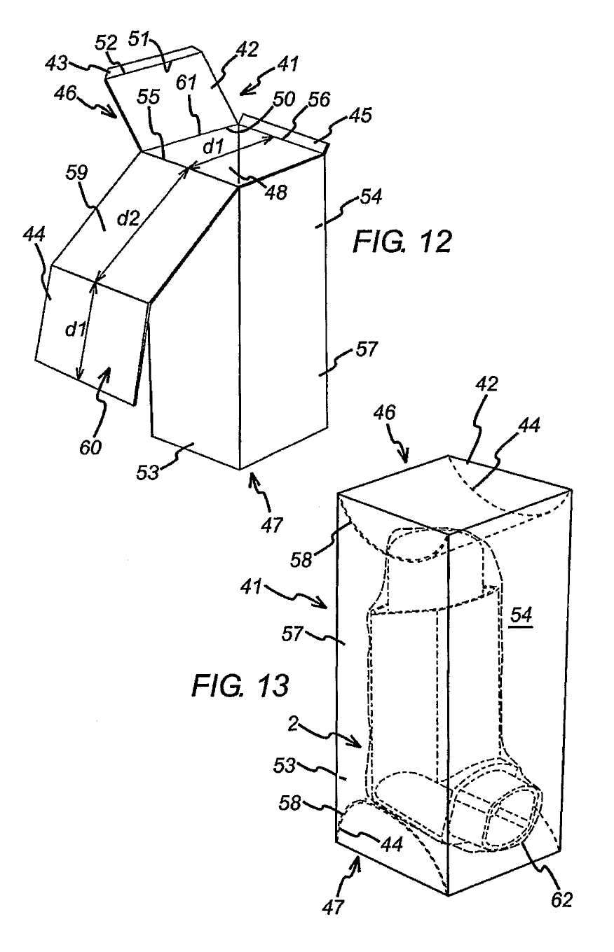

FIG. 12 shows an outer packaging for use with another, related, embodiment of the present invention. The outer packaging comprises a box (41) made from a resilient material, preferably cardboard. The top (46) and bottom (47) of the box (41) each comprise a lid (42), a lid flap (43) and side flaps (44, 45). At each end (46, 47) of the box (41), a first edge (61) of the lid (42) is attached to a first side (48) of the box (31) along a first fold line (50) and a lid flap (43) is provided at a second, opposite, side (51) of the lid (42), connected to the lid (42) along a second fold line (52). Second and third sides (53, 54) of the box (42), opposite one another and each adjacent said first side (48), are provided with side flaps (44, 45) attached to the box along fold lines (55, 56). When the lid (42) is moved to a closed position, it is folded along the first fold line (50) and its lid flap (43) is inserted into the box (41) between a fourth side (57), opposite the first side (48) of the box (41), and the side flaps (44, 45). The lid flap (42) may be provided with slits along the first fold line (50) that engage the side flaps (44, 45) and hold the lid (42) in its closed position.

Cardboard boxes according to the above description are common in the prior art. However boxes that are suitable for use as packages in the present embodiment additionally comprise at least one elongated flap. Preferably, one flap at each end is elongated. FIG. 12 shows a possible embodiment where one side flap (44) is elongated at the top end (46) of the box (41), however other flaps (43, 45) may be elongated in addition to, or in place of, this.

FIG. 13 shows a box (41) having an elongated side flap (44) at each end (46, 47) in use, containing a pMDI (2). The elongated flaps (44) are arched across the ends of the box (46, 47), creating elastically deformable, resilient cushions (58) across its top (46) and bottom (47). The box (41) and elongated flaps (44) are dimensioned such that the pMDI (2) is gripped between the cushions (58), and thus suspended inside the box (41). As well as substantially reducing movement of the pMDI (2) within the box (41), the cushions (58) dampen the effect on the pMDI (2) of any impacts the box (41) experiences.

The pMDI (2) may be inserted into an overwrap (62) before being inserted into the box (41), so that the overwrap (62) containing the pMDI (2) is held suspended between the cushions (58).

In preferred embodiments, the length of an elongated flap is greater than twice the width of the side on which it is mounted. In the example of FIG. 12, the elongated side flap (44) is of length d1+d2, where d1 is distance between the second and third sides (53, 54), and where d2>d1. In use, the extended flap (44) is folded across the top (46) of the box (41), underneath the lid (42), then a second portion (60) of the flap (44) is folded back along fold line. Since the length (d2) of the first portion (59) is greater than d1, the first portion (59) arches convexly into the box (41), forming a cushion (58). The depth of this arch can be chosen by varying length d2, which is preferably chosen such that the pMDI (2) is an interference fit between opposite cushions (58).

In a less advantageous embodiment, the elongated flap is of length d2 and is not folded back on itself, only arched between opposite sides. This embodiment is less advantageous since it weakens the structure of the box since the lid flap is no longer supported by the underlying extended side flap.

Although FIG. 13 illustrates an embodiment with an elongated side flap (44) at each end (56, 57) of the box (41), the lid flap (43) may be elongated instead. Furthermore, elongated flaps are not necessarily arched over the top (56) and bottom (57) of the box (4) and may be arched instead across the inner side walls (48, 53, 54, 57) of the box, so that the sides of the pMDI (2) are gripped horizontally. Finally a plurality of cushions (58) could be used to grip the pMDI (2) simultaneously in more than one dimension.

It will be understood that the present invention has been described above by way of example only and that the above description should not be taken to impose any limitation on the scope of the claims. The invention in its full spirit and scope is defined by the appended claims.

1: A package containing a dispenser and at least one support which suspends the dispenser in the package.

2: The package of claim 1, wherein the package has an outer package in which the dispenser is suspended by the at least one support.

3: The package of claim 1, wherein the dispenser is operated to dispense by the relative movement of its parts.

4: The package of claim 1, wherein the dispenser is an inhaler.

5: The package of claim 3, wherein the dispenser is a pMDI.

6: The package of claim 1, wherein the at least one support is made from a resilient material.

7: The package of claim 6 wherein the resilient material is cardboard.

8: The package of claim 1, wherein the at least one support comprises at least one insert.

9: The package of claim 1, wherein the at least one support is in a deformed configuration.

10: The package of claim 9, wherein the at least one support is biased to return to its non-deformed configuration.

11: The package of claim 9, wherein the at least one support in its deformed configuration has a generally semi-cylindrical form.

12: The package of claim 9, wherein the at least one support in its deformed configuration has a generally ‘V’ shaped cross section.

13: The package of claim 1, wherein the at least one support comprises a securing means for securing the dispenser to the support.

14: The package of claim 13 wherein the securing means comprises at least a shelf, on which the dispenser is located.

15: The package of claim 13 wherein the securing means comprising at last a tab engaging the dispenser.

16: The package of claim 10, wherein the at least one support comprises an end flap to one side of at least one end, wherein the end flip is secured to the opposite side of the at least one support, preventing the at least one support from adopting its non-deformed configuration.

17: The package of claim 16, wherein the at least one support comprises an end flap at both ends.

18: The package of claim 16, wherein the end flap is longer then the distance between the two sides of the at least one support and protrudes away from the support.

19: The package of claim 1, wherein the at least one support has a generally ‘Z’ shape.

20: The package of claim 1, wherein the at least one support comprises a window, wherein the dispenser extends through the window.

21: The package of claim 20, wherein the dispenser is an interference fit in the window.

22: The package of claim 19, wherein the at least one support further comprises at least one shelf which supports the dispenser.

23: The package of claim 22, wherein the at least one support comprises first and second supporting shelves at opposite ends of the dispenser.

24: The package of claim 1, wherein the at least one support comprises at least two flexible webs positioned on opposite sides of the dispenser.

25: The package of claim 2, wherein the at least one support comprises at least two flexible webs positioned on opposite sides of the dispenser and, wherein the opposite ends of each flexible web are positioned at opposite points on the inner surface of the outer package and the web has a length greater than the distance between theses two points, such that is arches between them.

26: The package of claim 25, wherein the webs arch towards the dispenser.

27: The package of claim 25, wherein the webs arch away from the dispenser.

28: The package of claim 24, wherein the dispenser is gripped between the webs.

29: The package of claim 24, wherein the webs are inserts.

30: The package of claim 24, wherein the package comprises a box having two or more elongated flaps.

31: The package of claim 30, wherein each web is formed by an elongated flap.

32: The package of claim 1, comprising a sheath in which the dispenser is enclosed, optionally suspendingly.

33: The package claim 32, wherein the sheath is heat-sealed.

34: The package of claim 32, wherein the support is inside the sheath.

35: The package of claim 32, wherein the support suspends the sheath with the dispenser inside the sheath.

36: A method of packing a dispenser, comprising:
providing a package and at least one support as defined in claim 1,
suspending the dispenser in the package with the at least one support; and
closing the package.

37-38. (canceled)
